(19) United States
(12) Patent Application Publication
(10) Pub. No.: US 2006/0178676 A1
(43) Pub. Date: Aug. 10, 2006

(54) Skin smoothing implement

(76) Inventors: Paul M. Anderson, Lincroft, NJ (US); Lisa F. Baumgarten, New York, NY (US); Antonette Bivona, Bayside, NY (US); John D. Butcher, Scotch Plains, NJ (US); Ingrid Yung-I Chen, New York, NY (US); Emily M. Cohen, New York, NY (US); Heath A. Doty, St. Louis, MO (US); Jeffery F. Feng, St. Louis, MO (US); Stacey Leigh Grabiner, Westfield, NJ (US); Jagne H. Lynch, St. Louis, MO (US); Bryce G. Rutter, St. Louis, MO (US); Heather S. Sopczynski, New York, NY (US)

Correspondence Address: Julie Blackburn;Revlon Consumer Products Corporation, 237 Park Avenue, New York, NY, 10017, US

(21) Appl. No.: 11/052,183

(22) Filed: Feb. 7, 2005

(51) Int. Cl. A61B 17/50

(52) U.S. Cl. 606/131

(57) ABSTRACT

A tool for abrading skin from keratinous surfaces comprising a closed loop of a certain circumference, said loop having an outer surface to which is affixed a continuous contoured abrasive surface covering at least a portion of the outer loop circumference, and a method for treating skin with such a tool.

### FIELD OF THE INVENTION

The invention is in the field of pedicure tools, more specifically those for use in abrading skin from feet, elbows, and other bodily areas that may suffer from rough skin.

### BACKGROUND OF THE INVENTION

There are a wide variety of implements available for use in abrading rough skin from the soles of the feet, elbows, or other areas. For example, the typical pumice stone is used for that purpose. Pumice stones may be in the shape of a rectangular block or smooth round stone, with the term “pumice” referring to the porous stone from which the implement is made. The user holds the pumice stone in the hand and uses it to abrade rough skin from the desired area. Often these types of pumice stones are hard to hold and manipulate. Further, these stones are not practical for users that have trouble reaching the area to be abraded due to disability or injury. Accordingly, implements manufacturers have made other types of abrading devices that are designed to overcome these disadvantages.

U.S. Design Pat. No. 416,648 is directed to a manicure/pedicure tool in the shape of triangular block with abrasive paper affixed to the three sides of the block. While the length of the triangular block gives the user a longer surface to leverage in attempting to abrade skin from areas such as the soles of the feet, the user must still grip the outer surface of the tool. As there is no handle or other gripping means, the user can easily lose control of the device. Moreover, as the sides of the block are planar, and the surface of the foot contoured, it is difficult to reach valleys in the soles of the feet or treat angled surfaces such as elbows.

U.S. Pat. Nos. D260,304 and 4,286,610 are directed to another type of skin abrading tool having a handle with a bottom planar surface where two different grades of abrasive paper are affixed to the surface. The handle enables the user to maintain a more secure grip on the tool, however because the abrasive surface is close, and parallel, to the handle it is still difficult for compromised users to abrade skin on difficult to reach areas. Also, the planar abrading surface makes it difficult to reach areas of the skin that are contoured.

U.S. Design Pat. No. 281,729 depicts another type of handled tool where the abrasive surface is a block set into a handled device. While the handle facilitates gripping, the abrasive surface is still too close to the handle to permit reaching difficult places. As with the other types of designs, the flat planar surface of the abrasive block makes it difficult to reach contoured areas on the soles of the feet or elbows, for example.

U.S. Pat. Nos. 4,366,328 and 4,211,246 are directed to a manicure buffing implement in the closed form with an internal channel where the user can extend the fingers through the channel to hold the implement, if desired.

U.S. Design Pat. No. 235,942 is directed to a brush suitable for cleaning in corners. The handle is in a generally circular closed configuration with a continuous brush surface spanning approximately forty percent of the handle circumference.

Each of these designs poses problems. While a number of the implements have handles, they do not appear to be ergonomic in configuration. The devices that have no handles are simple and inexpensive to manufacture but difficult to use or manipulate. Moreover, the devices that have long planar abrasive surfaces are not optimal for reaching contoured areas such as the soles of the feet or elbows.

Accordingly, there is a need for a skin abrading tool, preferably for pedicure use, that is easy to grip and manipulate, effective in abrading rough skin on contoured skin surfaces such as feet and elbows, and which enables the user to effectively treat difficult-to-reach areas.

It is an object of the invention to provide a skin abrading tool which is optimal for reaching all skin surfaces including those surfaces that are contoured.

It is a further object of the invention to provide a skin abrading tool that is ergonomically constructed to facilitate gripping and use.

It is a further object of the invention to provide a skin abrading tool that is easy to use, relatively inexpensive, easy to manufacture, and which provides optimal results.

### SUMMARY OF THE INVENTION

A tool for abrading skin from keratinous surfaces comprising a closed loop of a certain circumference, said loop having an outer surface to which is affixed a continuous contoured abrasive surface covering at least a portion of the outer loop circumference.

A method for abrading skin from contoured keratinous surfaces by treating the surface with a closed loop of a certain circumference having affixed to the outer surface thereof a continuous contoured abrasive surface covering at least a portion of the loop circumference.

### DETAILED DESCRIPTION OF DRAWINGS

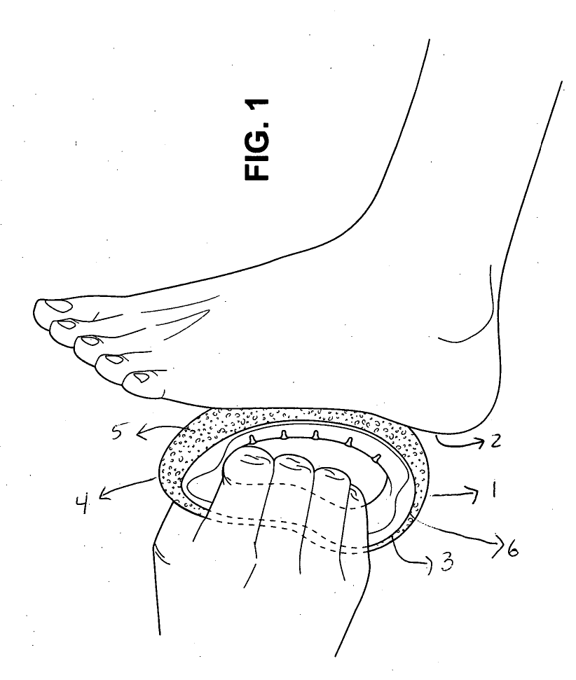

FIG. 1: depicts the tool of the invention being used to abrade skin from the sole of the foot.

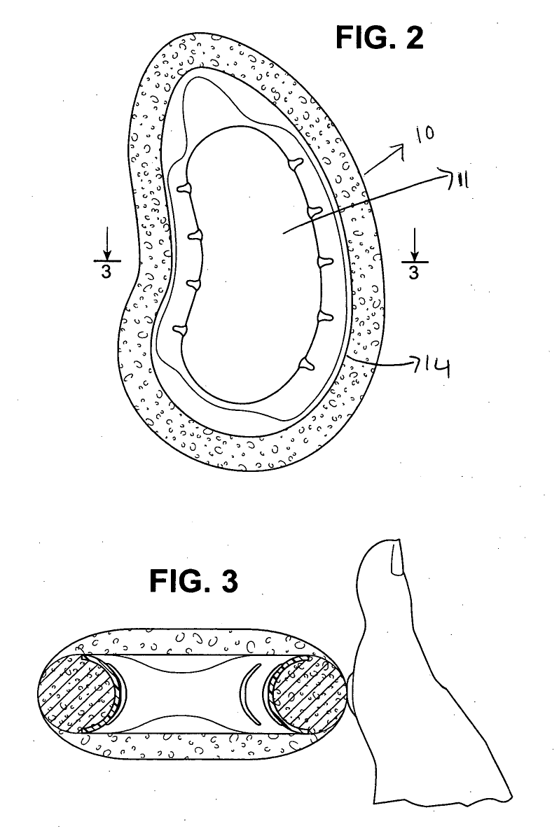

FIG. 2: depicts a side view of the tool of FIG. 1.

FIG. 2A: depicts a side view of the tool where the continuous abrasive surface covers a portion of the closed loop.

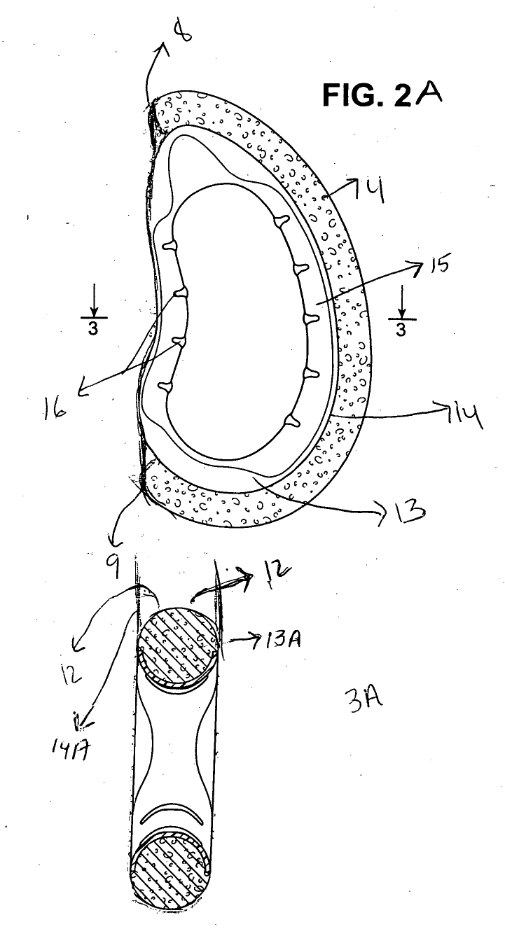

FIG. 3: depicts a cross-sectional view taken across 3-3 of FIG. 2, depicting the cross-sectional configuration of the tool showing the abrasive surface and gripping member.

FIG. 3A: depicts a side cross-sectional view taken across 3-3 of FIG. 2A, and illustrating the angle of the outer circumference of continuous abrasive surface.

### DETAILED DESCRIPTION

FIGS. 1 and 2 in general depict the tool 1 of the invention, which may be used to abrade skin from the soles of the feet 2, as well as other surfaces. The tool 1 is in the form of a closed loop 3 having a continuous abrasive surface 4. The term “continuous” means that the abrasive surface 4 is in one continuous section, which may extend the entire circumference 6 of the closed loop 3, or only a portion thereof. In the event where the continuous abrasive surface 4 extends only for a portion of the entire circumference 6 of the closed loop 3 as depicted in FIG. 2A, the abrasive surface 4 has one beginning 8 and one end 9 and there are no intermediate breaks in the continuous abrasive surface 4. The continuous abrasive surface 4 extends for at least a portion of the circumference 6 of closed loop 3, preferably at least about fifty percent of the circumference 6, more preferably about seventy five percent of the circumference. In the most preferred embodiment of the invention the continuous abrasive surface 4 extends for the entire circumference 6 of closed loop 3.

The continuous abrasive surface 4 may be made of any material that provides abrasion when rubbed or scraped against the desired keratinous surface. The abrasive material is preferably a porous synthetic or natural material that is sufficiently abrasive to be capable of abrading skin when scraped against the desired keratinous surface. Typical types of porous material include pumice (also known as lava froth), and other types of stones. It is preferred that the abrasive surface be made of a stone like pumice so that the entire continuous abrasive surface is in stone. Alternatively, it is possible to fashion the continuous abrasive surface out of a thermoplastic material such as foam or similar, and cover it with an abrasive coating that can be glued on, or the thermoplastic material can be covered with an abrasive cloth or paper, like sandpaper, to provide the necessary abrasive surface.

The continuous abrasive surface 4 covering closed loop 3 is contoured, meaning that it has sides and edges that are rounded in shape to as to better enable the user to treat hard-to-reach areas on the soles of the feet or elbows, for example. Such contoured edges are superior for treating skin on non-flat surfaces, as opposed to abrasive surfaces that are flat planar surfaces. In the latter case, the flat planar surfaces treat only the top surface of the sole of the foot, for example, with the depressions on the foot remaining untreated. The end result is that the skin is unevenly abraded and smoothened on the keratinous surface.

The closed loop 3 may have a variety of different configurations. Preferably, it is generally circular, oval, or kidney-bean shaped in circumference 10, with the inner space 11 being large enough to permit the user to extend the fingers through so that the closed loop 3 can be gripped when used to abrade skin on the desired keratinous surface.

Preferably the continuous abrasive surface 4 is contoured such that the angle 12, measured as depicted in FIG. 3A, ranges from 1 to 90 degrees, more preferably from about 1 to 45 degrees, more preferably 5 to 45 degrees. When the angle is in this range, the external surface of the continuous abrasive surface 4 is rounded, which in turn optimizes abrasion of hard-to-reach areas such as valleys in the soles of the feet or around the elbows. In general, such a rounded configuration also enables the continuous abrasive surface to better mold into skin peaks and depressions.

While it is not essential, preferably the closed loop has an inner surface gripping member 13. Gripping member 13 is designed to fit within space 11 and along internal circumference 14 of continuous abrasive surface 4 so that it is secured to closed loop 3 such that the user of the item can slide the fingers through space 11, grip the gripping member 13, and use the device in the intended manner. Preferably, gripping member 13 is made of synthetic or natural rubber or a similar synthetic thermoplastic material. Gripping member 13 may be made of synthetic thermoplastic material that is somewhat pliable so that the user's fingers depress the gripping member 13 so that it molds very slightly around the fingers. Gripping member has the same general circumference as closed loop 3, which may be circular, oval, or bean shaped. Alternatively, gripping member 13 may be of a more rigid thermoplastic material which is overlayered by a second, more pliable thermoplastic layer 15 that may, if desired, have slits 16 that are in the form of cuts or protrusions in the pliable thermoplastic layer 15. The purpose of slits 16 is to make the surface of pliable thermplastic layer 15 even more ergonomic and easy to grip by the user when it is used in the intended manner.

FIG. 3A is a cross-sectional view taken across 3-3 of FIG. 2 showing the continuous abrasive surface 4, gripping member 13, and pliable thermoplastic layer, in cross-section. The angle 12 formed between the cross-section 13A of the continuous abrasive surface 4, and a straight line 14A drawn parallel to the cross-section of the device, preferably ranges from 1 to 90 degrees, more preferably from 1 to 45 degrees, more preferably 5 to 45 degrees. This causes the curved sides of continuous abrasive surface 4 in cross-section 13A to better reach uneven skin surfaces.

The tool of the invention enables the user to better abrade rough skin from keratinous surfaces. Due to its ergonomic configuration it is easy to handle and easy to use.

We claim:

1. A tool for abrading skin from keratinous surfaces comprising a closed loop of a certain circumference, said loop having an outer surface to which is affixed a continuous contoured abrasive surface covering at least a portion of the outer loop circumference.

2. The tool of claim 1 wherein the continuous contoured abrasive surface covers more than about fifty percent of the loop outer surface circumference.

2. The tool of claim 1 wherein the continuous contoured abrasive surface covers more than about seventy five percent of the loop circumference.

3. The tool of claim 1 wherein the continuous contoured abrasive surface covers the entire loop circumference.

4. The tool of claim 1 wherein the abrasive surface is a porous synthetic or natural stone.

5. The tool of claim 4 wherein the abrasive surface is a porous natural stone.

6. The tool of claim 4 wherein the abrasive surface is pumice.

7. The tool of claim 1 wherein the loop is in a generally circular configuration.

8. The tool of claim 7 wherein the loop has an inner surface gripping member.

9. The tool of claim 8 wherein the inner surface gripping member is made of rubber.

10. The tool of claim 8 wherein the inner surface gripping member spans the entire circumference of the loop.

11. The tool of claim 8 wherein the inner surface gripping member has depressions in a portion thereof.

12. The tool of claim 1 where the abrasive surface is contoured at an angle ranging from about 1 to 90 degrees.

13. The tool of claim 1 wherein the abrasive surface is contoured at an angle ranging from about 5 to 45 degrees.

14. A method for abrading skin from contoured keratinous surfaces by treating the surface with a closed loop of a certain circumference having affixed to the outer surface thereof a continuous contoured abrasive surface covering at least a portion of the loop circumference.

15. The method of claim 14 wherein the abrasive surface is contoured at an angle ranging from about 1 to 90 degrees.

16. The method of claim 15 wherein the abrasive surface is contoured at an angle ranging from about 5 to 45 degrees.

17. The method of claim 14 wherein the continuous contoured abrasive surface covers at least about fifty percent of the loop circumference.

18. The method of claim 14 wherein the continuous contoured abrasive surface covers at least about seventy five percent of the loop circumference.

19. The method of claim 14 wherein the abrasive surface is a synthetic or natural porous stone.

20. The method of claim 19 wherein the abrasive surface is pumice.
